(19) United States
(12) Patent Application Publication
(10) Pub. No.: US 2025/0025111 A1
(43) Pub. Date: Jan. 23, 2025

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventor: Yuki WADA, Tokyo (JP)

(21) Appl. No.: 18/907,593

(22) Filed: Oct. 7, 2024

Related U.S. Application Data
(63) Continuation of application No. PCT/JP2023/013730, filed on Mar. 31, 2023.

(30) Foreign Application Priority Data
Apr. 18, 2022 (JP) 2022-068441

(51) Int. Cl. A61B 5/00; G16H 10/60; G16H 15/00; G16H 20/00

(52) U.S. Cl. CPC A61B 5/746; G16H 10/60; G16H 15/00; G16H 20/00

(57) ABSTRACT

An information processing apparatus includes at least one processor. The processor is configured to acquire a first medical document in which information on a disease recognized for a patient is described, and acquire a second medical document in which information on a treatment for the disease of the patient or information on course information on the disease of the patient is described. The processor is configured to issue, in a case in which an untreated disease, which is a disease not described in the second medical document, is present among the diseases described in the first medical document, an alarm indicating that the untreated disease is present.

### CROSS-REFERENCE TO RELATED APPLICATIONS

This application is a continuation application of International Application No. PCT/JP2023/013730, filed Mar. 31, 2023, the disclosure of which is incorporated herein by reference in its entirety. Further, this application claims priority from Japanese Patent Application No. 2022-068441, filed on Apr. 18, 2022, the disclosure of which is incorporated herein by reference in its entirety.

### BACKGROUND

### 1. Technical Field

The technology of the present disclosure relates to an information processing apparatus, an information processing method, and an information processing program.

### 2. Description of the Related Art

As a technology for preventing overlooking of matters described in an interpretation report, the following technology is known. For example, JP2020-38429A discloses a medical support device comprising a medical report acquisition unit that acquires a medical report, an importance degree determination unit that determines an importance degree of the medical report, a distribution destination determination unit that determines a distribution destination of an alert related to the medical report by using the importance degree, and a distribution unit that distributes the alert to the distribution destination determined by the distribution destination determination unit.

JP2019-204189A discloses an interpretation report management device comprising a creation unit that extracts a predetermined character string from a first interpretation report created by an interpretation doctor in response to an interpretation request from a clinical doctor and creates a second interpretation report in which a checkbox is provided at a position corresponding to the extracted predetermined character string in the first interpretation report, a check determination unit that determines whether or not a check is put in all the checkboxes provided in the second interpretation report, and a reception unit that receives a display end request of the second interpretation report in a case in which the check is put in all the checkboxes provided in the second interpretation report.

### SUMMARY

Information on a disease extracted from a medical image, such as a plain X-ray image, a computed tomography (CT) image, a mammography image, or a magnetic resonance imaging (MRI) image, is described in an interpretation report. A doctor performs appropriate treatment for the disease described in the interpretation report. In a case in which the disease described in the interpretation report is overlooked, a serious incident, such as a delay in treatment for the disease and a worsening of symptoms, may occur.

The technology of the present disclosure has been made in view of the above-described point, and an object thereof is to raise awareness for overlooking a disease described in a medical document such as an interpretation report.

The technology of the present disclosure relates to an information processing apparatus including at least one processor. The processor is configured to acquire a first medical document in which information on a disease recognized for a patient is described, and acquire a second medical document in which information on a treatment for the disease of the patient or information on course information on the disease of the patient is described. The processor is configured to issue, in a case in which an untreated disease, which is a disease not described in the second medical document, is present among the diseases described in the first medical document, an alarm indicating that the untreated disease is present.

According to the technology of the present disclosure, it is possible to raise awareness for overlooking the disease described in the medical document such as the interpretation report.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a medical information management system according to an embodiment of the technology of the present disclosure;

FIG. 2 is a diagram showing an example of a hardware configuration of an information processing server according to the embodiment of the technology of the present disclosure;

FIG. 3 is a functional block diagram showing an example of a functional configuration of the information processing server according to the embodiment of the technology of the present disclosure;

FIG. 4 is a diagram showing an example of a list according to the embodiment of the technology of the present disclosure;

FIG. 5 is a diagram showing an example of alarm display according to the embodiment of the technology of the present disclosure;

FIG. 6 is a flowchart showing an example of a flow of processing performed by executing an information processing program via a CPU according to the embodiment of the technology of the present disclosure;

FIG. 7 is a diagram showing an example of an importance degree table according to the embodiment of the technology of the present disclosure;

FIG. 8 is a flowchart showing an example of a flow of the processing performed by executing the information processing program via the CPU according to the embodiment of the technology of the present disclosure;

FIG. 9 is a diagram showing an example of the importance degree table according to the embodiment of the technology of the present disclosure;

FIG. 10 is a functional block diagram showing an example of the functional configuration of the information processing server according to the embodiment of the technology of the present disclosure;

FIG. 11 is a flowchart showing an example of a flow of the processing performed by executing the information processing program via the CPU according to the embodiment of the technology of the present disclosure;

FIG. 12 is a flowchart showing an example of a flow of the processing performed by executing the information processing program via the CPU according to the embodiment of the technology of the present disclosure;

FIG. 13 is a flowchart showing an example of a flow of the processing performed by executing the information processing program via the CPU according to the embodiment of the technology of the present disclosure; and

FIG. 14 is a flowchart showing an example of a flow of the processing performed by executing the information processing program via the CPU according to the embodiment of the technology of the present disclosure.

### DETAILED DESCRIPTION

Hereinafter, examples of embodiments of the technology of the present disclosure will be described with reference to the accompanying drawings. It should be noted that the same or equivalent components and parts in the respective drawings are denoted by the same reference numerals, and the duplicated description will be omitted.

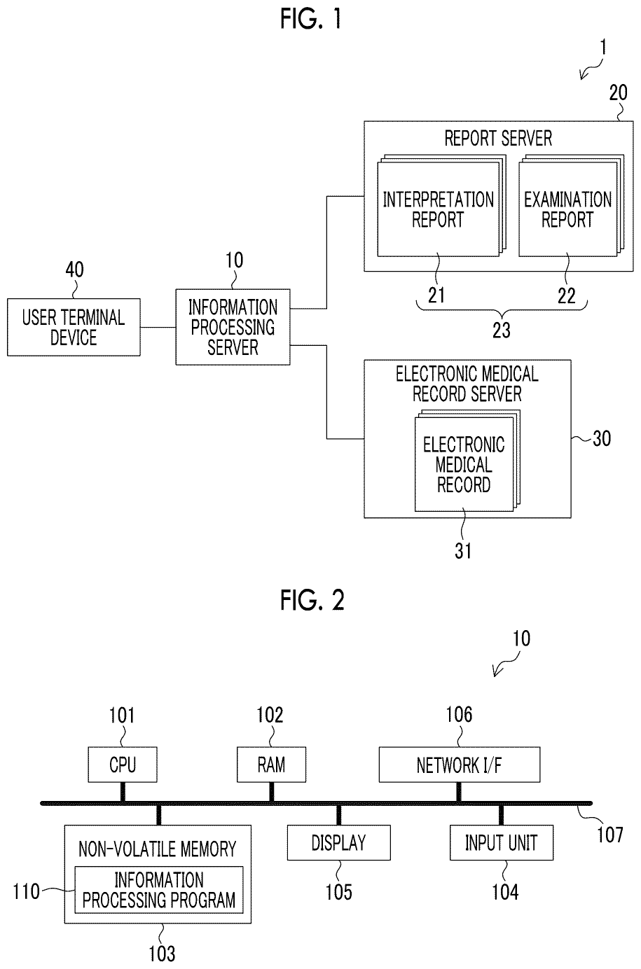

FIG. 1 is a diagram showing an example of a configuration of a medical information management system 1 according to the embodiment of the technology of the present disclosure. The medical information management system 1 has a configuration including an information processing server 10, a report server 20, an electronic medical record server 30, and a user terminal device 40.

The report server 20 is a server computer comprising a storage medium that stores medical documents such as an interpretation report 21 and an examination report 22. In the interpretation report 21, for example, information on a disease extracted from medical images, such as a plain X-ray image, a CT image, a mammography image, or an MRI image, is described. In the examination report 22, for example, information on a result of an examination performed on a tissue or an organ collected from a patient is described. The interpretation report 21 and the examination report 22 are created for each patient. A plurality of interpretation reports 21 and a plurality of examination reports 22 may be created for one patient. It should be noted that, in a case in which the distinction between the interpretation report 21 and the examination report 22 is not made or the distinction between the interpretation report 21 are collectively referred to, the interpretation report 21 and the examination report 22 are simply referred to as “report 23” in the following description. The report 23 is an example of a “first medical document” according to the technology of the present disclosure.

The electronic medical record server 30 is a server computer comprising a storage medium that stores the electronic medical record 31 created for each patient. The electronic medical record 31 includes information on a treatment for a disease of the patient or course information on the disease of the patient. Examples of the “information on the treatment” include subjective information, objective information, assessment, plan, a medication status, an examination result, an examination order, and the like for the disease.

A doctor who creates the electronic medical record 31 registers the disease described in the report 23 as a disease that needs to be treated for the patient by using a registration system (not shown). The registered disease is recorded in the electronic medical record 31 through system linkage. The disease includes a positive disease that is determined in a definitive manner and a suspected disease (false positive disease) that is not determined in a definitive manner but is suspected as being positive. Information indicating whether the disease is the positive disease or the suspected disease is assigned to each disease described in the electronic medical record 31. The electronic medical record 31 is an example of a “second medical document” according to the technology of the present disclosure.

In a case in which an untreated disease, which is a disease not described in the electronic medical record 31, is present among the diseases described in the report 23 for a specific patient designated by the user terminal device 40, the information processing server 10 issues an alarm indicating that the untreated disease is present. Among the diseases described in the report 23, the disease that is not described in the electronic medical record 31 is presumed as a disease that is not recognized by the doctor as a disease that needs to be treated, and is handled as the untreated disease in the present system. Since the presence of the untreated disease may be caused by the overlooking of the disease described in the report 23, the information processing server 10 issues the alarm to the user terminal device 40 in a case in which the untreated disease is present. The information processing server 10 is an example of an “information processing apparatus” according to the technology of the present disclosure.

The user terminal device 40 is a computer intended to be used by a doctor. The user terminal device 40 inquires of the information processing server 10 about the presence or absence of untreated disease for the specific patient. The patient who is an inquiry target is designated by the user terminal device 40. The information processing server 10 determines the presence or absence of the untreated disease for the designated patient in response to the inquiry from the user terminal device 40, and issues the alarm to the user terminal device 40 in a case in which the untreated disease is present. The alarm, which is issued from the information processing server 10, is displayed on the display (not shown) of the user terminal device 40.

FIG. 2 is a diagram showing an example of a hardware configuration of the information processing server 10. The information processing server 10 includes a central processing unit (CPU) 101, a random access memory (RAM) 102, a non-volatile memory 103, an input device 104, a display 105, and a network interface 106. These pieces of hardware are connected to a bus 107.

The display 105 is, for example, a liquid crystal display or a light emitting diode (LED) display. The input device 104 includes, for example, a keyboard and a mouse, and may include a proximity input device such as a touch panel display, a voice input device such as a microphone, and a contactless input device such as a camera or a sensor. The network interface 106 is an interface for connecting the information processing server 10 to the report server 20, the electronic medical record server 30, and the user terminal device 40 via a network.

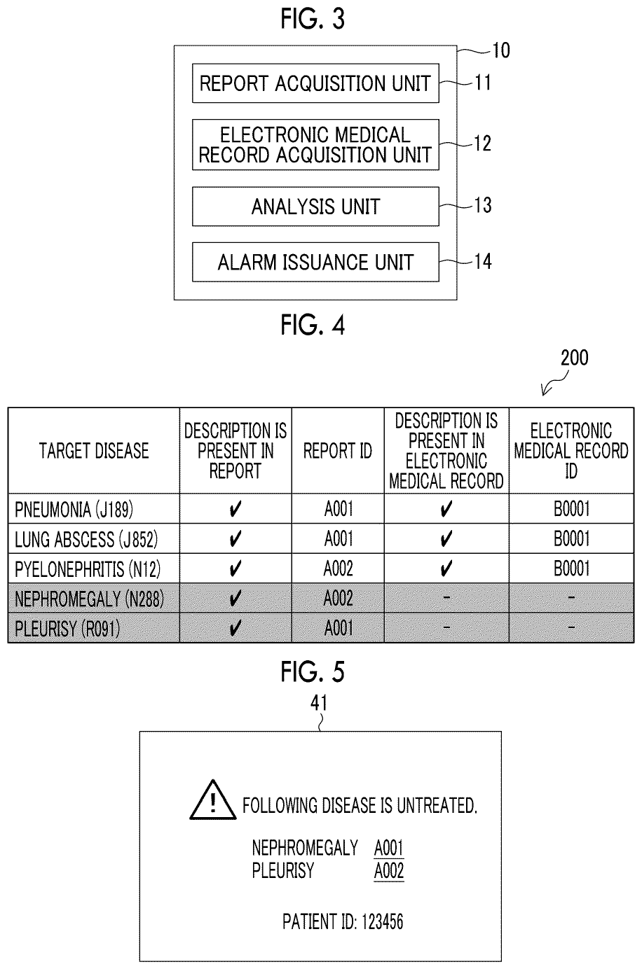

The non-volatile memory 103 is a non-volatile storage medium such as a hard disk and a flash memory. The non-volatile memory 103 stores an information processing program 110. The RAM 102 is a work memory for the CPU 101 to execute processing. The CPU 101 loads the information processing program 110 stored in the non-volatile memory 103 into the RAM 102, and executes the processing according to the information processing program 110. The CPU 101 is an example of a “processor” according to the technology of the present disclosure. FIG. 3 is a functional block diagram showing an example of a functional configuration of the information processing server 10. The information processing server 10 includes a report acquisition unit 11, an electronic medical record acquisition unit 12, an analysis unit 13, and an alarm issuance unit 14. The CPU 101 executes the information processing program 110, whereby the CPU 101 functions as the report acquisition unit 11, the electronic medical record acquisition unit 12, the analysis unit 13, and the alarm issuance unit 14.

The report acquisition unit 11 accesses the report server 20 to acquire the report 23 related to the specific patient from among the reports 23 stored in the storage medium provided in the report server 20. The specific patient is designated by the user terminal device 40. Patient identification information, such as a patient identification (ID), is assigned to the interpretation report 21 and the examination report 22. The report acquisition unit 11 transmits a report acquisition request including the patient identification information (patient ID) to the report server 20. The report server 20 extracts the report to which the patient identification information (patient ID) related to the acquisition request is assigned, from among the reports 23 stored in the storage medium provided in the report server 20, and transmits the extracted report to the information processing server 10. The report acquisition unit 11 acquires the report 23 transmitted from the report server 20. It should be noted that, in a case in which a plurality of reports 23 are created for one patient, all of the reports 23 for the patient are acquired.

The report server 20 may comprise a report database (not shown) in which the patient identification information (patient ID) and identification information (report ID) of the interpretation report 21 and the examination report 22 are associated with each other. In such a case, the report server 20 may extract the report 23 related to the specific patient by using the report database.

The electronic medical record acquisition unit 12 accesses the electronic medical record server 30 to acquire the electronic medical record 31 related to the specific patient from among the electronic medical records 31 stored in the storage medium provided in the electronic medical record server 30. The patient identification information (patient ID) is assigned to the electronic medical record 31. The electronic medical record acquisition unit 12 transmits an electronic medical record acquisition request including the patient identification information (patient ID) to the electronic medical record server 30. The electronic medical record server 30 extracts the electronic medical record 31 to which the patient identification information (patient ID) related to the acquisition request is assigned, from among the electronic medical records 31 stored in the storage medium provided in the electronic medical record server 30, and transmits the extracted electronic medical record 31 to the information processing server 10. The electronic medical record acquisition unit 12 acquires the electronic medical record 31 transmitted from the electronic medical record server 30. It should be noted that, in a case in which a plurality of electronic medical records 31 are created for one patient, all of the electronic medical records 31 for the patient are acquired.

The electronic medical record server 30 may comprise an electronic medical record database (not shown) in which the patient identification information (patient ID) and the identification information (electronic medical record ID) of the electronic medical record 31 are associated with each other. In such a case, the electronic medical record server 30 may extract the electronic medical record 31 related to the specific patient by using the electronic medical record database.

The analysis unit 13 analyzes the text described in the report 23 acquired by the report acquisition unit 11, extracts a description part including a name or a type of the disease described in the report, and specifies the disease described in the report based on the extracted description part.

Similarly, the analysis unit 13 analyzes the text described in the electronic medical record 31 acquired by the electronic medical record acquisition unit 12, extracts a description part including a name or a type of the disease described in the electronic medical record 31, and specifies the disease described in the electronic medical record 31 based on the extracted description part. It should be noted that, in a case in which the electronic medical record 31 stores, for each electronic medical record 31, the disease in a form for specifying the disease, including the name or the type of the disease, the text analysis or the like is not required.

The alarm issuance unit 14 determines whether or not the untreated disease, which is the disease not described in the electronic medical record 31, is present among the diseases described in the report 23 and specified by the analysis unit 13. In a case in which the alarm issuance unit 14 determines that the untreated disease is present, the alarm issuance unit 14 issues the alarm indicating that the untreated disease is present.

The alarm issuance unit 14 generates a list 200 (see FIG. 4) indicating the presence or absence of descriptions in the report 23 and the electronic medical record 31 for target diseases, including the disease described in the report 23 and the disease described in the electronic medical record 31, based on the analysis result by the analysis unit 13. The target disease may be classified according to the International Classification of Diseases, 10th Revision (ICD-10).

In the list 200, a check mark indicating that the description of the disease is present in the report 23 is assigned for each target disease. According to the list 200 shown in FIG. 4, it is shown that the description about “pneumonia”, “lung abscess”, “pyelonephritis”, “nephromegaly”, and “pleurisy” is present in the report 23. In addition, each of the diseases described in the report 23 is associated with the identification information (report ID) of the report 23 including the description.

Similarly, in the list 200, a check mark indicating that the description of the disease is present in the electronic medical record 31 is assigned for each target disease. According to the list 200 shown in FIG. 4, it is shown that the descriptions about “pneumonia”, “lung abscess”, and “pyelonephritis” are present in the electronic medical record 31. In addition, each of the diseases described in the electronic medical record 31 is associated with the identification information (electronic medical record ID) of the electronic medical record 31 including the description.

The alarm issuance unit 14 determines whether or not the untreated disease, which is the disease not described in the electronic medical record 31, is present among the diseases described in the report 23 based on the list 200. In addition, the alarm issuance unit 14 specifies a name or a type of the untreated disease. According to the list 200 shown in FIG. 4, the alarm issuance unit 14 specifies that “nephromegaly” and “pleurisy” are the untreated diseases. In such a case, the alarm issuance unit 14 issues the alarm indicating that the untreated disease is present. The alarm issuance unit 14 may include, in the alarm, information indicating “nephromegaly” and “pleurisy” specified as the untreated diseases. In addition, the alarm issuance unit 14 may present information required to access the report 23 in which the untreated disease is described. For example, the alarm issuance unit 14 may present, along with the alarm, a hyperlink for accessing the report ID “A002” of the report 23 in which “nephromegaly” is described and the report ID “A001” of the report 23 in which “pleurisy” is described. In addition, the list 200 may be presented along with the alarm. The alarm issued by the alarm issuance unit 14 is transmitted to the user terminal device 40. A display 41 of the user terminal device 40 that has received the alarm performs, for example, display as shown in FIG. 5.

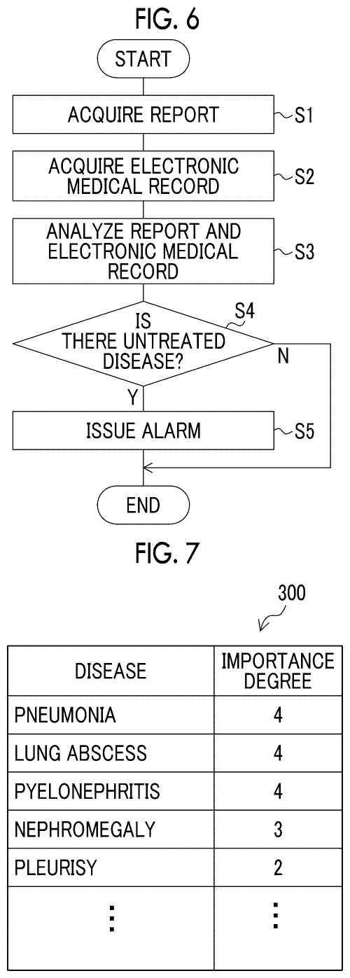

FIG. 6 is a flowchart showing an example of a flow of processing performed by executing the information processing program 110 via the CPU 101 according to the embodiment of the technology of the present disclosure. The information processing program 110 is executed, for example, in a case in which there is the inquiry about the presence or absence of the untreated disease for the specific patient is issued from the user terminal device 40.

In step S1, the report acquisition unit 11 accesses the report server 20 to acquire the report 23 related to the specific patient from among the reports 23 stored in the storage medium provided in the report server 20. Specifically, the report acquisition unit 11 transmits the report acquisition request including the patient identification information (patient ID) to the report server 20. The report server 20 extracts the report to which the patient identification information (patient ID) related to the acquisition request is assigned, from among the reports 23 stored in the storage medium provided in the report server 20, and transmits the extracted report to the information processing server 10. The report acquisition unit 11 acquires the report 23 transmitted from the report server 20.

In step S2, the electronic medical record acquisition unit 12 accesses the electronic medical record server 30 to acquire the electronic medical record 31 related to the specific patient from among the electronic medical records 31 stored in the storage medium provided in the electronic medical record server 30. Specifically, the electronic medical record acquisition unit 12 transmits the electronic medical record acquisition request including the patient identification information (patient ID) to the electronic medical record server 30. The electronic medical record server 30 extracts the electronic medical record to which the patient identification information (patient ID) related to the acquisition request is assigned, from among the electronic medical records 31 stored in the storage medium provided in the electronic medical record server 30, and transmits the extracted electronic medical record 31 to the information processing server 10. The electronic medical record acquisition unit 12 acquires the electronic medical record 31 transmitted from the electronic medical record server 30. It should be noted that a case is described in which the report 23 is acquired before the electronic medical record 31 is acquired, but the present disclosure is not limited to this. The report 23 may be acquired after the electronic medical record 31 is acquired, or the acquisition of the report 23 and the acquisition of the electronic medical record 31 may be performed in parallel.

In step S3, the analysis unit 13 analyzes the text described in the report 23 acquired in step S1, and extracts the description part including the name or the type of the disease described in the report 23. The analysis unit 13 specifies the disease described in the report 23 based on the extracted description part. Similarly, the analysis unit 13 analyzes the text described in the electronic medical record 31 acquired in step S2, and extracts the description part including the name or the type of the disease described in the electronic medical record 31. The analysis unit 13 specifies the disease described in the electronic medical record 31 based on the extracted description part.

In step S4, the alarm issuance unit 14 determines whether or not the untreated disease, which is the disease not described in the electronic medical record 31, is present among the diseases described in the report 23 and specified in step S3. In a case in which a determination is made that the untreated disease is present, the processing proceeds to step S5. In a case in which a determination is made that the untreated disease is absent, the present routine ends.

In step S5, the alarm issuance unit 14 issues the alarm indicating that the untreated disease is present. The alarm issued by the alarm issuance unit 14 is transmitted to the user terminal device 40. The display 41 of the user terminal device 40 that has received the alarm performs, for example, display as shown in FIG. 5.

As described above, the information processing server 10 according to the embodiment of the technology of the present disclosure acquires the report 23 in which the information on the disease recognized for the patient is described. In addition, the information processing server 10 acquires the electronic medical record 31 in which the information on the treatment for the disease of the patient or the course information on the disease of the patient is described. In a case in which the untreated disease, which is the disease not described in the electronic medical record 31, is present among the diseases described in the report 23, the information processing server 10 issues the alarm indicating that the untreated disease is present.

As described above, the doctor who creates the electronic medical record 31 registers the disease described in the report 23 as the disease that needs to be treated for the patient by using the registration system (not shown). The registered disease is recorded in the electronic medical record 31 through system linkage. In a case in which the disease described in the report 23 is overlooked, the disease may not be recorded in the electronic medical record 31 and may remain unrecognized as the treatment target. That is, the disease described in the report 23 is recognized as the treatment target in a case in which the disease is described in the electronic medical record 31, but there is a concern that the disease is not recognized as the treatment target when the disease is not described in the electronic medical record 31.

With the information processing server 10 according to the present embodiment, in a case in which the untreated disease, which is the disease not described in the electronic medical record 31, is present among the diseases described in the report 23, the alarm indicating that the untreated disease is present is issued. That is, in a case in which it is presumed that the disease described in the report 23 is overlooked, notification of such a situation is issued. Therefore, it is possible to raise awareness for the overlooking of the disease described in the report 23.

In addition, the information processing server 10 specifies the untreated disease by analyzing the text described in the report 23 and the electronic medical record 31, and presents the information indicating the specified untreated disease (for example, the name or the type of the disease) along with the alarm. As a result, the doctor, who is the user, can understand the untreated disease, and thus it is possible to promptly perform a required treatment on the untreated disease.

In addition, the information processing server 10 presents information (for example, the report ID or the hyperlink) required to access the report 23 in which the untreated disease is described. As a result, it is possible to quickly access the information on the untreated disease.

It should be noted that, in the present embodiment, a case is described in which the alarm is issued in a case in which the untreated disease is present for the specific patient designated by the user terminal device 40. However, the technology of the present disclosure is not limited to this aspect. The information processing server 10 may determine whether or not the untreated disease is present for each patient managed in the medical information management system 1, and may issue the alarm for all of the corresponding patients in a case in which the untreated disease is present. In addition, the patient who is the target of the alarm to be issued may be registered for each user, and the alarm may be issued in a case in which the untreated disease is present for the registered patient. For example, in the information processing server 10, user identification information of the user and patient identification information of the patient under the user's care may be registered in association with each other, the user who operates the user terminal device 40 may be specified by login processing using an ID and a password for each user between the user terminal device 40 and the information processing server 10, the patient identification information of the patient associated with the user identification information of the specified user may be specified, and the alarm may be issued in a case in which the untreated disease is present for the specified patient. For example, by registering the patient under the care for each doctor or for each medical department to which the doctor belongs, in a case in which the doctor logs in, the alarm may be issued in a case in which the untreated disease is present for the patient under the doctor's care, or the alarm may be issued in a case in which the untreated disease is present for the patient under the care of the medical department to which the doctor belongs. Further, the user may be able to register the patient who is the target of the alarm to be issued, for each user.

In addition, in the present embodiment, a case is described in which the information processing server 10 issues the alarm by executing the information processing program 110, but the user terminal device 40 may issue the alarm by executing the information processing program 110. In such a case, the user terminal device 40 functions as an “information processing apparatus” according to the technology of the present disclosure. In such a case, the user terminal device 40 may acquire the report 23 and the electronic medical record 31 via the information processing server 10. Further, the user terminal device 40 may directly access the report server 20 and the electronic medical record server 30 to acquire the report 23 and the electronic medical record 31. In such a case, since the user terminal device 40 also has a function as the information processing server 10, the information processing server 10 can be omitted in the medical information management system 1.

### Second Embodiment

The information processing server 10 according to the first embodiment described above always issues the alarm in a case in which the untreated disease is present. On the other hand, the information processing server 10 according to the second embodiment issues the alarm based on an importance degree set for the untreated disease.

The non-volatile memory 103 of the information processing server 10 according to the present embodiment stores an importance degree table 300 as shown in FIG. 7. The importance degree table 300 is a table in which the disease and the importance degree are associated with each other. The importance degree is set in consideration of, for example, a risk of severity and a risk of death of the disease.

In a case in which the untreated disease is present, the alarm issuance unit 14 specifies the importance degree set for the untreated disease by referring to the importance degree table 300 stored in the non-volatile memory 103. The alarm issuance unit 14 issues the alarm in a case in which the importance degree specified for the untreated disease is equal to or higher than a threshold value, and does not issue the alarm in a case in which the importance degree is lower than the threshold value. In a case in which the threshold value related to the importance degree is, for example, 3, the alarm is issued in a case in which the disease specified as the untreated disease is “nephromegaly” with the importance degree of 3, while the alarm is not issued in a case in which the disease specified as the untreated disease is “pleurisy” with the importance degree of 2.

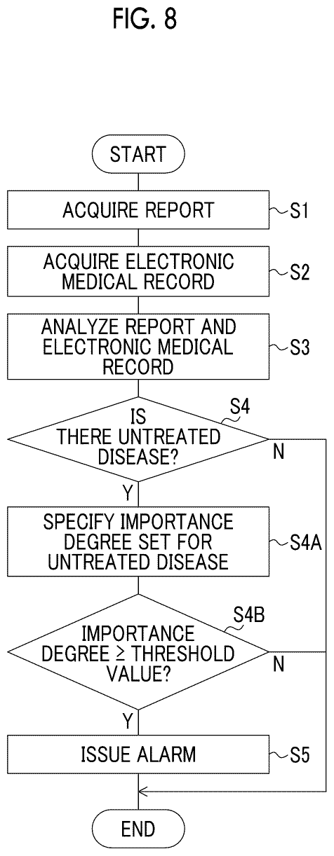

FIG. 8 is a flowchart showing an example of a flow of the processing performed by the CPU 101 according to the present embodiment executing the information processing program 110. In the present flowchart, steps S4A and S4B are added to the flowchart shown in FIG. 6. Hereinafter, only steps different from the flowchart shown in FIG. 6 will be described, and the description of the other steps will be omitted.

In step S4A, the alarm issuance unit 14 specifies the importance degree for the untreated disease related to the determination in step S4 by referring to the importance degree table 300 stored in the non-volatile memory 103.

In step S4B, the alarm issuance unit 14 determines whether or not the importance degree specified in step S4A is equal to or higher than the threshold value. In a case in which a determination is made that the importance degree is equal to or higher than the threshold value, the processing proceeds to step S5. In step S5, the alarm issuance unit 14 issues the alarm indicating that the untreated disease is present. On the other hand, in a case in which a determination is made that the importance degree is lower than the threshold value, the present routine ends.

As described above, the information processing server 10 according to the present embodiment issues the alarm based on the importance degree set for the untreated disease. As a result, it is possible to avoid the frequent occurrence of the alarms for the untreated disease with a low importance degree, and it is possible to issue the alarm more effectively to raise awareness.

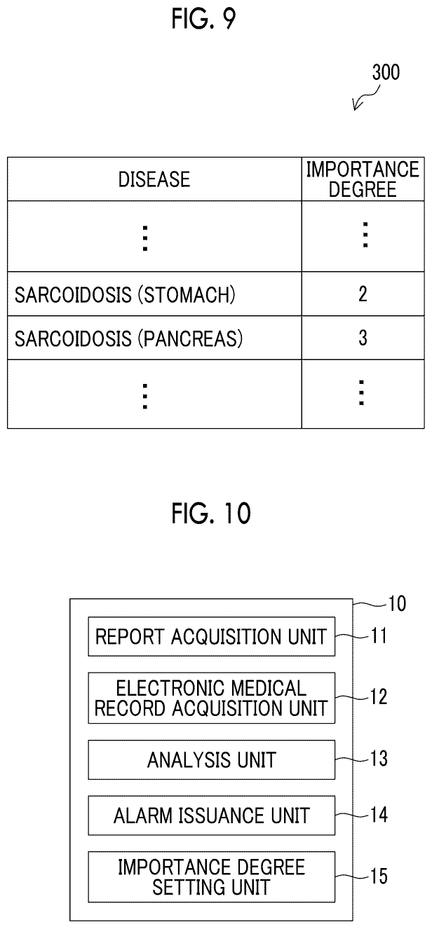

In the importance degree table 300, the importance degree associated with each disease may be set based on the type of the organ related to the disease. FIG. 9 shows a case in which, in the importance degree table 300, the importance degree of 2 is set for sarcoidosis that has developed in the stomach, and the importance degree of 3 is set for sarcoidosis that has developed in the pancreas. In this way, different importance degrees may be set to the same disease that develops in different organs. According to the importance degree table 300 shown in FIG. 9, in a case in which the threshold value related to the importance degree is, for example, 3, the alarm is issued for “sarcoidosis that has developed in the pancreas” specified as the untreated disease, but the alarm is not issued for “sarcoidosis that has developed in the stomach” specified as the untreated disease. It should be noted that it is possible to specify the organ related to the disease specified as the untreated disease through the text analysis of the corresponding report 23.

### Third Embodiment

In the second embodiment described above, the importance degree set for the untreated disease is a fixed value set in the importance degree table 300. In the third embodiment, the importance degree set for the untreated disease is set by the information processing server 10.

FIG. 10 is a functional block diagram showing an example of a functional configuration of the information processing server 10 according to the third embodiment of the technology of the present disclosure. The information processing server 10 according to the third embodiment includes an importance degree setting unit 15. The importance degree setting unit 15 specifies a progression degree or an urgency degree for the untreated disease based on the description about the untreated disease in the report 23. For example, in a case in which the untreated disease is a polyp, the importance degree setting unit 15 may specify the progression degree or the urgency degree of the polyp based on a size of the polyp described in the report 23. The importance degree setting unit 15 sets the importance degree for the untreated disease based on the specified progression degree or the specified urgency degree. The importance degree setting unit 15 sets a relatively high importance degree for the disease with a relatively high progression degree or the urgency degree. The setting of the importance degree may be performed, for example, by correcting a standard value of the importance degree set in advance for each disease based on the progression degree or the urgency degree.

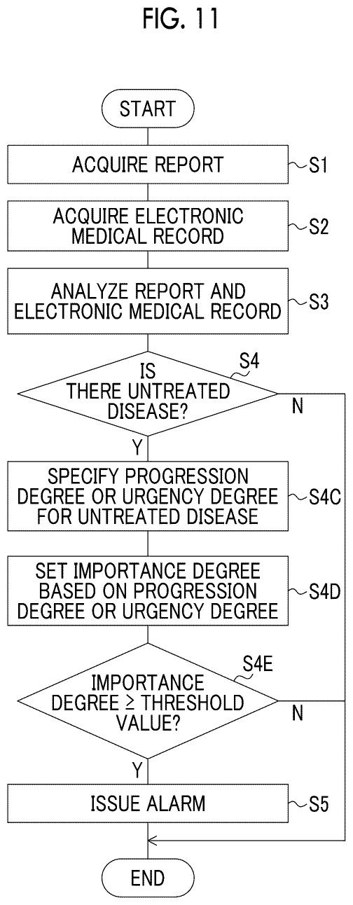

FIG. 11 is a flowchart showing an example of a flow of the processing performed by the CPU 101 according to the present embodiment executing the information processing program 110. In the present flowchart, steps S4C, S4D, and S4E are added to the flowchart shown in FIG. 6. Hereinafter, only steps different from the flowchart shown in FIG. 6 will be described, and the description of the other steps will be omitted.

In step S4C, the importance degree setting unit 15 specifies the progression degree or the urgency degree for the untreated disease related to the determination in step S4. The importance degree setting unit 15 specifies the progression degree or the urgency degree of the untreated disease based on the description about the untreated disease in the report 23.

In step S4D, the importance degree setting unit 15 sets the importance degree for the untreated disease based on the progression degree or the urgency degree specified in step S4C.

In step S4E, the alarm issuance unit 14 determines whether or not the importance degree set in step S4D is equal to or higher than the threshold value. In a case in which a determination is made that the importance degree is equal to or higher than the threshold value, the processing proceeds to step S5. In step S5, the alarm issuance unit 14 issues the alarm indicating that the untreated disease is present. On the other hand, in a case in which a determination is made that the importance degree is lower than the threshold value, the present routine ends.

With the information processing server 10 according to the present embodiment, it is possible to avoid the frequent occurrence of the alarms for the untreated disease with a low progression degree or a low urgency degree, and it is possible to issue the alarm more effectively to raise awareness. With the information processing server 10 according to the present embodiment, for example, an aspect can be realized in which the alarm is issued for the polyp having a size of 10 mm while the alarm is not issued for the polyp having a size of 3 mm.

### Fourth Embodiment

The information processing server 10 according to the third embodiment described above sets the importance degree of the untreated disease based on the specified progression degree or the specified urgency degree of the untreated disease. On the other hand, the information processing server 10 according to the fourth embodiment sets the importance degree of the untreated disease based on a relevance between an organ related to a treatment target disease, which is the disease described in the electronic medical record 31, and the organ related to the untreated disease. It should be noted that the disease that is described in the electronic medical record 31 is presumed as a disease that is recognized by the doctor as the disease that needs to be treated, and is handled as the treatment target disease in the present system.

The importance degree setting unit 15 according to the present embodiment specifies the organ related to the treatment target disease based on the description about the treatment target disease in the electronic medical record 31. For example, the importance degree setting unit 15 specifies “kidney”, which is an organ related to “chronic kidney disease”, based on the description about “chronic kidney disease”, which is the treatment target disease, in the electronic medical record 31. Similarly, the importance degree setting unit 15 specifies the organ related to the untreated disease based on the description about the untreated disease in the report 23. For example, the importance degree setting unit 15 specifies “kidney”, which is the organ related to “kidney cyst”, based on the description about “kidney cyst”, which is the untreated disease, in the report 23. In addition, “liver”, which is the organ associated with “liver cyst”, is specified based on the description about “liver cyst”, which is the untreated disease, in the report 23.

The importance degree setting unit 15 sets a lower importance degree for the untreated disease in a case in which the organ related to the treatment target disease and the organ related to the untreated disease are the same as each other, than in a case in which these organs are different from each other. For example, since “kidney”, which is the organ related to “chronic kidney disease” that is the treatment target disease, and “liver”, which is the organ related to “liver cyst” that is the untreated disease, are different organs, the importance degree setting unit 15 sets the importance degree of 3 for “liver cyst” that is the untreated disease. On the other hand, since “kidney”, which is the organ related to “chronic kidney disease” that is the treatment target disease, and “kidney”, which is the organ related to “kidney cyst” that is the untreated disease, are the same organ, the importance degree setting unit 15 sets the importance degree of 2 for “kidney cyst” that is the untreated disease.

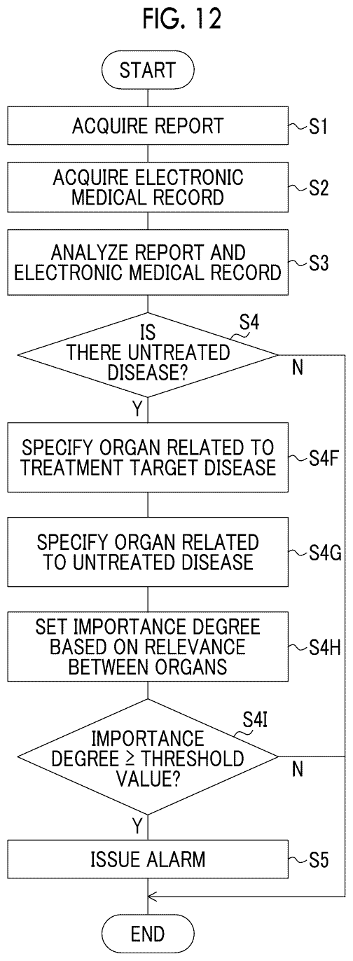

FIG. 12 is a flowchart showing an example of a flow of the processing performed by the CPU 101 according to the present embodiment executing the information processing program 110. In the present flowchart, steps S4F, S4G, S4H, and S4I are added to the flowchart shown in FIG. 6. Hereinafter, only steps different from the flowchart shown in FIG. 6 will be described, and the description of the other steps will be omitted.

In step S4F, the importance degree setting unit 15 specifies the organ related to the treatment target disease based on the description about the treatment target disease in the electronic medical record 31.

In step S4G, the importance degree setting unit 15 specifies the organ related to the untreated disease based on the description about the untreated disease in the report 23.

In step S4H, the importance degree setting unit 15 sets the importance degree for the untreated disease based on a relevance between the organ related to the treatment target disease specified in step S4F and the organ related to the untreated disease specified in step S4G. Specifically, a lower importance degree is set for the untreated disease in a case in which the organ related to the treatment target disease and the organ related to the untreated disease are the same as each other, than in a case in which these organs are different from each other. For example, the importance degree set for the untreated disease in a case in which the organ related to the treatment target disease and the organ related to the untreated disease are different from each other may be determined in advance as the standard value, and the importance degree of the untreated disease in a case in which the organs are the same as each other may be set by correcting the standard value in a case in which these organs are the same as each other.

In step S4I, the alarm issuance unit 14 determines whether or not the importance degree set in step S4H is equal to or higher than the threshold value. In a case in which a determination is made that the importance degree is equal to or higher than the threshold value, the processing proceeds to step S5. In step S5, the alarm issuance unit 14 issues the alarm indicating that the untreated disease is present. On the other hand, in a case in which a determination is made that the importance degree is lower than the threshold value, the present routine ends.

Since some treatment has already been performed on the organ related to the treatment target disease, it is considered that there is no high necessity to issue the alarm for the untreated disease discovered in the same organ as the organ related to the treatment target disease. With the information processing server 10 according to the present embodiment, the issuance of the alarm is restricted for the untreated disease discovered in the same organ as the organ related to the treatment target disease.

### Fifth Embodiment

The information processing server 10 according to the second to fourth embodiments described above issues an alarm based on the importance degree set for the untreated disease. On the other hand, the information processing server 10 according to the fifth embodiment issues an alarm based on a comparison result between a first importance degree set for the untreated disease and a second importance degree set for the treatment target disease that is the disease described in the electronic medical record 31.

The non-volatile memory 103 of the information processing server 10 according to the present embodiment stores an importance degree table 300 as shown in FIG. 7. The importance degree table 300 is a table in which the disease and the importance degree are associated with each other. The importance degree is set in consideration of, for example, the risk of severity and the risk of death of the disease.

In a case in which the untreated disease is present, the alarm issuance unit 14 specifies the first importance degree set for the untreated disease by referring to the importance degree table 300 stored in the non-volatile memory 103. In addition, the alarm issuance unit 14 specifies the second importance degree set for the treatment target disease, which is the disease described in the electronic medical record 31, by referring to the importance degree table 300. The alarm issuance unit 14 issues the alarm in a case in which the first importance degree is equal to or higher than the second importance degree, and does not issue the alarm in a case in which the first importance degree is lower than the second importance degree.

For example, the alarm is issued in a case in which the disease specified as the untreated disease is “lung abscess” with the importance degree of 4 and the disease specified as the treatment target disease is “nephromegaly” with the importance degree of 3, but the alarm is not issued in a case in which the disease specified as the untreated disease is “pleurisy” with the importance degree of 2 and the disease specified as the treatment target disease is “nephromegaly” with the importance degree of 3.

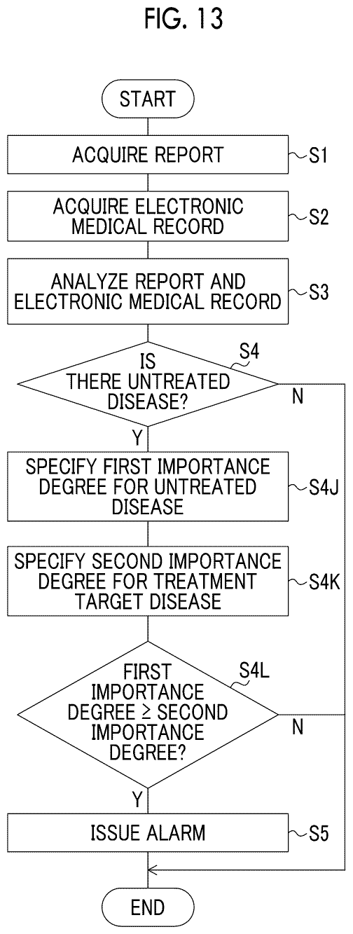

FIG. 13 is a flowchart showing an example of a flow of the processing performed by the CPU 101 according to the present embodiment executing the information processing program 110. In the present flowchart, steps S4J, S4K, and S4L are added to the flowchart shown in FIG. 6. Hereinafter, only steps different from the flowchart shown in FIG. 6 will be described, and the description of the other steps will be omitted.

In step S4J, the alarm issuance unit 14 specifies the first importance degree for the untreated disease related to the determination in step S4 by referring to the importance degree table 300 stored in the non-volatile memory 103.

In step S4K, the alarm issuance unit 14 specifies the treatment target disease described in the electronic medical record 31, and specifies the second importance degree for the treatment target disease by referring to the importance degree table 300 stored in the non-volatile memory 103.

In step S4L, the alarm issuance unit 14 determines whether or not the first importance degree specified in step S4J is equal to or higher than the second importance degree specified in step S4K. In a case in which a determination is made that the first importance degree is equal to or higher than the second importance degree, the processing proceeds to step S5. In step S5, the alarm issuance unit 14 issues the alarm indicating that the untreated disease is present. On the other hand, in a case in which a determination is made that the first importance degree is lower than the second importance degree, the present routine ends.

It is considered that, since a large amount of medical resources is required for the treatment target disease with a high importance degree, it is preferable to lower priority of the treatment for the untreated disease with a relatively low importance degree. With the information processing server 10 according to the present embodiment, in a case in which the treatment target disease with a relatively high importance degree is present, the issuance of the alarm indicating that the untreated disease is present is restricted.

### Sixth Embodiment

In the fifth embodiment described above, the first importance degree set for the untreated disease and the second importance degree set for the treatment target disease are fixed values set in the importance degree table 300. In the sixth embodiment, the first importance degree and the second importance degree are set by the information processing server 10.

The information processing server 10 according to the sixth embodiment includes the importance degree setting unit 15 (see FIG. 10) as in the third embodiment. The importance degree setting unit 15 specifies the progression degree or the urgency degree for the untreated disease based on the description about the untreated disease in the report 23. The importance degree setting unit 15 sets the first importance degree for the untreated disease based on the specified progression degree or the specified urgency degree. The setting of the first importance degree may be performed, for example, by correcting a standard value of the importance degree set in advance for each disease based on the progression degree or the urgency degree.

In addition, the importance degree setting unit 15 specifies the progression degree or the urgency degree for the treatment target disease based on the description about the treatment target disease in the electronic medical record 31. The importance degree setting unit 15 sets the second importance degree for the treatment target disease based on the specified progression degree or the specified urgency degree. The setting of the second importance degree may be performed, for example, by correcting a standard value of the importance degree set in advance for each disease based on the progression degree or the urgency degree.

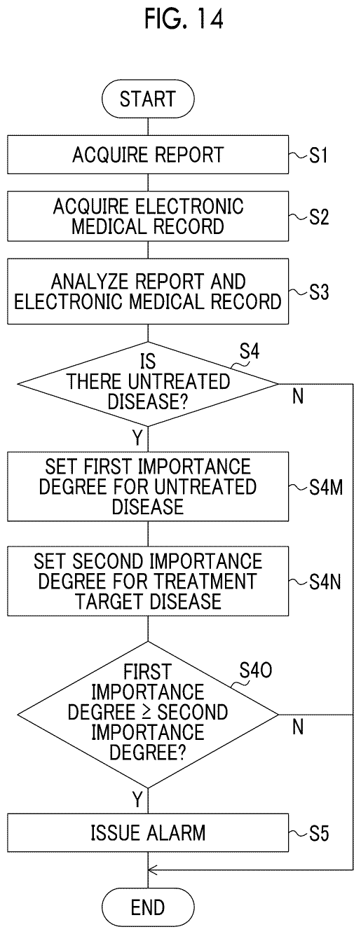

FIG. 14 is a flowchart showing an example of a flow of the processing performed by the CPU 101 according to the present embodiment executing the information processing program 110. In the present flowchart, steps S4M, S4N, and S40 are added to the flowchart shown in FIG. 6. Hereinafter, only steps different from the flowchart shown in FIG. 6 will be described, and the description of the other steps will be omitted.

In step S4M, the importance degree setting unit 15 specifies the progression degree or the urgency degree for the untreated disease related to the determination in step S4. The importance degree setting unit 15 specifies the progression degree or the urgency degree of the untreated disease based on the description about the untreated disease in the report 23. The importance degree setting unit 15 sets the first importance degree for the untreated disease based on the specified progression degree or the specified urgency degree.

In step S4N, the importance degree setting unit 15 specifies the treatment target disease described in the electronic medical record 31 and specifies the progression degree or the urgency degree for the treatment target disease. The importance degree setting unit 15 specifies the progression degree or the urgency degree of the treatment target disease based on the description in the electronic medical record 31 in which the treatment target disease is described. The importance degree setting unit 15 sets the second importance degree for the treatment target disease based on the specified progression degree or the specified urgency degree.

In step S40, the alarm issuance unit 14 determines whether or not the first importance degree set in step S4M is equal to or higher than the second importance degree set in step S4N. In a case in which a determination is made that the first importance degree is equal to or higher than the second importance degree, the processing proceeds to step S5. In step S5, the alarm issuance unit 14 issues the alarm indicating that the untreated disease is present. On the other hand, in a case in which a determination is made that the first importance degree is lower than the second importance degree, the present routine ends.

In the above description, a case is described in which the second importance degree for the treatment target disease is set based on the progression degree or the urgency degree, but the importance degree setting unit 15 may set the second importance degree based on the number of the treatment target diseases or a treatment period. For example, the second importance degree may be set to be higher as the number of the treatment target diseases is larger and as the treatment period is longer. That is, a higher importance degree is set for the treatment target disease that requires more medical resources. As a result, in a case in which there is the treatment target disease that requires more medical resources, the issuance of the alarm indicating that the untreated disease is present is restricted. Examples of a case in which the number of the treatment target diseases is large include multiple organ dysfunction syndrome. In addition, examples of the disease with a long treatment period include cancer that requires a significant amount of time for treatment.

Here, in a case in which the alarm is issued based on a comparison result between the first importance degree set for the untreated disease and the second importance degree set for the treatment target disease, there is a concern that the issuance of the alarm may be excessively restricted in a case in which the first importance degree and the second importance degree are uniformly determined in accordance with the type of the disease, the progression degree, or the urgency degree.

For example, in a case in which an attempt is made to always issue the alarm in a case in which the untreated disease is “fracture”, it is considered that the importance degree (first importance degree) for “fracture” is set to the highest rank. On the other hand, in a case in which “fracture” is the treatment target disease and the importance degree (second importance degree) is set to the highest rank, only the untreated disease with the importance degree set to the highest rank is the alarm issuance target, and the alarm cannot be issued for the untreated disease with an importance degree lower than the highest rank. For example, although it is preferable to issue the alarm for “gastritis” that is the untreated disease with a lower importance degree than “fracture” that is the treatment target disease, the alarm is not issued.

In that respect, the importance degree setting unit 15 may set the first importance degree set for the untreated disease in a case in which the specific disease is the untreated disease and the second importance degree set for the treatment target disease in a case in which the specific disease is the treatment target disease, to be different from each other. More specifically, the first importance degree set for the untreated disease in a case in which the specific disease is the untreated disease may be set to be lower than the second importance degree set for the treatment target disease in a case in which the specific disease is the treatment target disease. For example, the importance degree setting unit 15 may set “4” as the first importance degree set for “fracture” in a case in which “fracture” is the untreated disease, and may set “3” as the second importance degree set for “fracture” in a case in which “fracture” is the treatment target disease. As a result, even in a case in which “fracture” is the treatment target disease, it is possible to issue the alarm for “gastritis” that is the untreated disease with the importance degree of 3. Examples of the “specific disease” in which different importance degrees are set between the untreated disease and the treatment target disease include a disease in which a risk of deterioration is high in a case of being left untreated, a disease in which early treatment is required, and a disease in which remarkable recovery is expected by the treatment.

In each of the above-described embodiments, for example, as the hardware structure of the processing units that execute various types of processing, such as the report acquisition unit 11, the electronic medical record acquisition unit 12, the analysis unit 13, the alarm issuance unit 14, and the importance degree setting unit 15, various processors shown below can be used. The various processors include, in addition to a CPU and a GPU which are general-purpose processors that execute software (program) and function as various processing units as described above, a programmable logic device (PLD) which is a processor of which a circuit configuration can be changed after manufacture, such as an FPGA, and a dedicated electric circuit which is a processor of which a circuit configuration is designed for exclusive use in order to execute specific processing, such as an application specific integrated circuit (ASIC).

One processing unit may be configured by one of the various processors or may be configured by combining two or more processors of the same type or different types (for example, by combining a plurality of FPGAs or combining a CPU and an FPGA). A plurality of processing units may be configured by one processor.

A first example of the configuration in which the plurality of processing units are configured by one processor is a form in which one processor is configured by combining one or more CPUs and the software and this processor functions as the plurality of processing units, as represented by computers such as a client and a server. A second example thereof is a form in which a processor that realizes the function of the entire system including the plurality of processing units by one integrated circuit (IC) chip is used, as represented by a system on chip (SoC) or the like. In this way, various processing units are configured by one or more of the various processors as the hardware structure.

Further, the hardware structure of the various processors is, more specifically, an electric circuit (circuitry) in which circuit elements such as semiconductor elements are combined.

Moreover, in the above-described embodiments, an aspect is described in which the information processing program 110 is stored (installed) in advance in the non-volatile memory 103, but the technology of the present disclosure is not limited to this. The information processing program 110 may be provided in a form of being recorded in the recording medium such as a compact disc read only memory (CD-ROM), a digital versatile disc read only memory (DVD-ROM), and a universal serial bus (USB) memory. The information processing program 110 may be provided in a form being downloaded from an external device via a network. That is, the program (that is, a program product) described in the present embodiment may be provided using the recording medium or may be distributed from an external computer.

In regard to the first to sixth embodiments, the following supplementary notes will be further disclosed.

### (Supplementary Note 1)

An information processing apparatus including: at least one processor, in which the processor is configured to: acquire a first medical document in which information on a disease recognized for a patient is described; acquire a second medical document in which information on a treatment for a disease of the patient or course information on the disease of the patient is described; and issue, in a case in which an untreated disease, which is a disease not described in the second medical document, is present among the diseases described in the first medical document, an alarm indicating that the untreated disease is present. (Supplementary Note 2)

The information processing apparatus according to supplementary note 1, in which the processor is configured to specify the untreated disease by analyzing text described in the first medical document and text described in the second medical document, and present information indicating the specified untreated disease.

### (Supplementary Note 3)

The information processing apparatus according to supplementary note 1 or 2, in which the processor is configured to present information required to access the first medical document in which the untreated disease is described.

### (Supplementary Note 4)

The information processing apparatus according to any one of supplementary notes 1 to 3, in which the processor is configured to issue the alarm based on an importance degree set for the untreated disease.

### (Supplementary Note 5)

The information processing apparatus according to supplementary note 4, in which the importance degree is set based on a type of an organ related to the untreated disease.

### (Supplementary Note 6)

The information processing apparatus according to supplementary note 4 or 5, in which the processor is configured to specify a progression degree or an urgency degree of the untreated disease from the first medical document, and set the importance degree based on the specified progression degree or the specified urgency degree. (Supplementary Note 7)

The information processing apparatus according to supplementary note 4, in which the processor is configured to set the importance degree based on a relevance between an organ related to a treatment target disease, which is the disease described in the second medical document, and an organ related to the untreated disease. (Supplementary Note 8)

The information processing apparatus according to supplementary note 7, in which the processor is configured to lower the importance degree in a case in which the organ related to the treatment target disease and the organ related to the untreated disease are the same as each other than in a case in which these organs are different from each other.

### (Supplementary Note 9)

The information processing apparatus according to any one of supplementary notes 1 to 3, in which the processor is configured to issue the alarm based on a comparison result between a first importance degree set for the untreated disease and a second importance degree set for a treatment target disease which is the disease described in the second medical document. (Supplementary Note 10)

The information processing apparatus according to supplementary note 9, in which the processor is configured to set the first importance degree and the second importance degree based on the progression degree or the urgency degree of the treatment target disease and the untreated disease.

### (Supplementary Note 11)

The information processing apparatus according to supplementary note 9 or 10, in which the processor is configured to set the second importance degree based on the number of the treatment target diseases or a treatment period.

### (Supplementary Note 12)

The information processing apparatus according to supplementary note 9 or 10, in which the processor is configured to set a first importance degree set for the untreated disease in a case in which a specific disease is the untreated disease and a second importance degree set for the treatment target disease in a case in which the specific disease is the treatment target disease, to be different from each other.

### (Supplementary Note 13)

An information processing method including: via at least one processor provided in an information processing apparatus, acquiring a first medical document in which information on a disease recognized for a patient is described; acquiring a second medical document in which information on a treatment for a disease of the patient or course information on the disease of the patient is described; and issuing, in a case in which an untreated disease, which is a disease not described in the second medical document, is present among the diseases described in the first medical document, an alarm indicating that the untreated disease is present.

### (Supplementary Note 14)

An information processing program causing at least one processor provided in an information processing apparatus to execute a process including: acquiring a first medical document in which information on a disease recognized for a patient is described; acquiring a second medical document in which information on a treatment for a disease of the patient or course information on the disease of the patient is described; and issuing, in a case in which an untreated disease, which is a disease not described in the second medical document, is present among the diseases described in the first medical document, an alarm indicating that the untreated disease is present.

It should be noted that the disclosure of JP2022-068441 filed on Apr. 18, 2022 is incorporated in the present specification in its entirety by reference. All of the documents, the patent applications, and the technical standards described in the present specification are incorporated herein by reference to the same extent as in a case in which each document, each patent application, and each technical standard are specifically and individually described by being incorporated in the present specification by reference.

What is claimed is:

1. An information processing apparatus comprising:
at least one processor,
wherein the processor is configured to:
acquire a first medical document in which information on a disease recognized for a patient is described;
acquire a second medical document in which information on a treatment for a disease of the patient or course information on the disease of the patient is described; and
issue, in a case in which an untreated disease, which is a disease not described in the second medical document, is present among the diseases described in the first medical document, an alarm indicating that the untreated disease is present.

2. The information processing apparatus according to claim 1,
wherein the processor is configured to specify the untreated disease by analyzing text described in the first medical document and text described in the second medical document, and present information indicating the specified untreated disease.

3. The information processing apparatus according to claim 1,
wherein the processor is configured to present information required to access the first medical document in which the untreated disease is described.

4. The information processing apparatus according to claim 1,
wherein the processor is configured to issue the alarm based on an importance degree set for the untreated disease.

5. The information processing apparatus according to claim 4,
wherein the importance degree is set based on a type of an organ related to the untreated disease.

6. The information processing apparatus according to claim 4,
wherein the processor is configured to specify a progression degree or an urgency degree of the untreated disease from the first medical document, and set the importance degree based on the specified progression degree or the specified urgency degree.

7. The information processing apparatus according to claim 4,
wherein the processor is configured to set the importance degree based on a relevance between an organ related to a treatment target disease, which is the disease described in the second medical document, and an organ related to the untreated disease.

8. The information processing apparatus according to claim 7,
wherein the processor is configured to lower the importance degree in a case in which the organ related to the treatment target disease and the organ related to the untreated disease are the same as each other than in a case in which these organs are different from each other.

9. The information processing apparatus according to claim 1,
wherein the processor is configured to issue the alarm based on a comparison result between a first importance degree set for the untreated disease and a second importance degree set for a treatment target disease which is the disease described in the second medical document.

10. The information processing apparatus according to claim 9,
wherein the processor is configured to set the first importance degree and the second importance degree based on the progression degree or the urgency degree of the treatment target disease and the untreated disease.

11. The information processing apparatus according to claim 9,
wherein the processor is configured to set the second importance degree based on the number of the treatment target diseases or a treatment period.

12. The information processing apparatus according to claim 9,
wherein the processor is configured to set a first importance degree set for the untreated disease in a case in which a specific disease is the untreated disease and a second importance degree set for the treatment target disease in a case in which the specific disease is the treatment target disease, to be different from each other.

13. An information processing method comprising:
via at least one processor provided in an information processing apparatus,
acquiring a first medical document in which information on a disease recognized for a patient is described;
acquiring a second medical document in which information on a treatment for a disease of the patient or course information on the disease of the patient is described; and
issuing, in a case in which an untreated disease, which is a disease not described in the second medical document, is present among the diseases described in the first medical document, an alarm indicating that the untreated disease is present.

14. A non-transitory computer-readable storage medium storing an information processing program causing at least one processor provided in an information processing apparatus to execute a process comprising:
acquiring a first medical document in which information on a disease recognized for a patient is described;
acquiring a second medical document in which information on a treatment for a disease of the patient or course information on the disease of the patient is described; and
issuing, in a case in which an untreated disease, which is a disease not described in the second medical document, is present among the diseases described in the first medical document, an alarm indicating that the untreated disease is present.
